(19) United States
(12) Patent Application Publication
(10) Pub. No.: US 2012/0197395 A1
(43) Pub. Date: Aug. 2, 2012

(54) HYBRID SOLID-FLEXIBLE PASSING PIN AND ANTERIOR CRUCIATE LIGAMENT REPAIR USING THE PIN

(76) Inventor: Jeffrey H. Berg, Ashburn, VA (US)

(21) Appl. No.: 13/361,307

(22) Filed: Jan. 30, 2012

Related U.S. Application Data
(60) Provisional application No. 61/437,783, filed on Jan. 31, 2011.

(51) Int. Cl. A61F 2/08; A61B 17/16

(52) U.S. Cl. 623/13.14; 606/80

(57) ABSTRACT

A unitary hybrid solid-flexible wire pin with a leading portion composed of a solid, substantially rigid member and a trailing portion composed of a flexible component, the leading portion having a free first end and a second end, the trailing portion having a free first end and a second end, wherein the second end of the trailing portion and the second end of the leading portion are fixedly coupled to create the unitary hybrid solid-flexible wire pin. The unitary hybrid solid-flexible wire pin is used as a passing pin for tunnel reaming in anterior cruciate ligament repair.

### CROSS REFERENCE TO RELATED APPLICATIONS

This application claims the benefit of U.S. Provisional Patent Application Ser. No. 61/437,783, filed Jan. 31, 2011, entitled “HYBRID SOLID-FLEXIBLE PIN AND ANTERIOR CRUCIATE LIGAMENT REPAIR USING THE PIN”.

### BACKGROUND OF THE INVENTION

1. Field of the Invention

The invention relates to an apparatus and associated technique for anterior cruciate ligament repair.

2. Description of the Related Art

Standard Beath pins have been used for years to assist in (Anterior Cruciate Ligament) ACL femoral tunnel reaming. Traditionally, these ACL reconstructions have been performed using a transtibial technique. In particular, Beath pins are commonly used in anterior cruciate ligament reconstruction to pass a suture. In recent years, femoral tunnel reaming through an anteromedial portal has become more popular and now is widely used.

Although anteromedial femoral drilling allows for independent ideal femoral tunnel placement, it has been known to lead to potential complications and proficiency with this technique is dependent on a steep learning curve. Among the described complications associated with this technique are:
medial femoral chondral injury from inadvertent reamer contact; and
permanent pin deformity following repositioning (flexion & extension) of the knee and subsequent reamer-pin jamming, pin breakage and/or development of intra-articular metallic pin shavings.

In order to prevent these potential complications, a number of techniques have been developed. Among these, is the use of a Nitinol, that is, a nickel titanium shape memory alloy, flexible wire as a guide-wire following development of the pilot hole with a standard Beath pin. Due to this wire's ability to be temporarily deformed without permanent damage, this technique has the advantage of allowing the reamer to be passed over a wire and through the anteromedial portal well anterior to the medial femoral condyle and away from its chondral surface. Additionally, deep knee flexion and subsequent extension, as is required during anteromedial femoral tunnel reaming, does not lead to permanent deformation of the Beath pin.

The use of this wire, however, is not ideal. Due to its flexible nature, as well as its blunt tip, this wire cannot be used to drill the initial pilot hole and, therefore, an additional pin and step must be used. In addition, present Nitinol wires do not have eyelets or passing loops and therefore they cannot be used as passing pins. As a result, a second Beath pin and step are likewise required.

As such, improved techniques and instruments are required.

### SUMMARY OF THE INVENTION

It is, therefore, an object of the present invention to provide a unitary hybrid solid-flexible wire pin including a leading portion composed of a solid, substantially rigid member and a trailing portion composed of a flexible component. The leading portion has a free first end and a second end, and the trailing portion has a free first end and a second end. The second end of the trailing portion and the second end of the leading portion are fixedly coupled to create the unitary hybrid solid-flexible wire pin.

It is also an object of the present invention to provide a unitary hybrid solid-flexible wire pin wherein the second end of the trailing portion has an eyelet or a passing loop therein.

It is another object of the present invention to provide a unitary hybrid solid-flexible wire pin wherein the trailing portion is provided with distance markings thereon.

It is a further object of the present invention to provide a unitary hybrid solid-flexible wire pin wherein the distance markings are provided at 0.5 cm intervals.

It is also an object of the present invention to provide a unitary hybrid solid-flexible wire pin wherein the distance markings are placed at 1 cm intervals with a notch and 0.5 intervals with a dot.

It is another object of the present invention to provide a unitary hybrid solid-flexible wire pin wherein the trailing portion is composed of a nickel titanium shape memory alloy.

It is a further object of the present invention to provide a unitary hybrid solid-flexible wire pin wherein the leading portion and the trailing portion are joined by melding or by a press-fit male/female arrangement.

It is also an object of the present invention to provide a unitary hybrid solid-flexible wire pin wherein the free end of the leading end is a sharp tip.

It is another object of the present invention to provide a method for femoral tunnel reaming through an anteromedial portal during anterior cruciate ligament repair using an unitary hybrid solid-flexible wire pin. The unitary hybrid solid-flexible wire pin comprises a leading portion composed of a solid, substantially rigid member and a trailing portion composed of a flexible component. The leading portion has a free first end and a second end, and the trailing portion has a free first end and a second end. The second end of the trailing portion and the second end of the leading portion are fixedly coupled to create the unitary hybrid solid-flexible wire pin. The method is achieved by advancing the free end of the leading portion of the unitary hybrid solid-flexible wire pin through the anteromedial portal to develop a femoral pilot hole; further advancing the unitary hybrid solid-flexible wire pin in a retrograde manner until the trailing portion is inserted to a depth greater than a desired femoral tunnel length; passing a reamer over the unitary hybrid solid-flexible wire pin and through the anteromedial portal; deforming the unitary hybrid solid-flexible wire pin to allow the reamer to clear a medial femoral condoyle while hyperflexing a knee; removing the reamer from the femoral tunnel and extending the knee; and temporarily deforming the unitary hybrid solid-flexible wire pin to enable the reamer to be directed away from the femoral condoyle and out of the anteromedial portal.

It is a further object of the present invention to provide a method further comprising the step of passing a suture loop into an aperture in the trailing portion of the unitary solid-flexible wire pin and thereby into the femoral tunnel for graft passage while removing the unitary hybrid solid-flexible wire pin.

It is also object of the present invention to provide a method wherein the step of advancing the unitary hybrid solid-flexible wire pin in a retrograde manner until the trailing portion is inserted to a depth greater than a desired femoral tunnel length is achieved by use of distance markings located on the trailing portion of the unitary hybrid solid-flexible wire pin.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1, 2 and 3 are perspective view of the present hybrid solid-flexible wire pin.

FIGS. 4 to 9 show various steps associated with femoral tunnel reaming through an anteromedial portal employing the present hybrid solid-flexible wire pin during anterior cruciate ligament repair.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

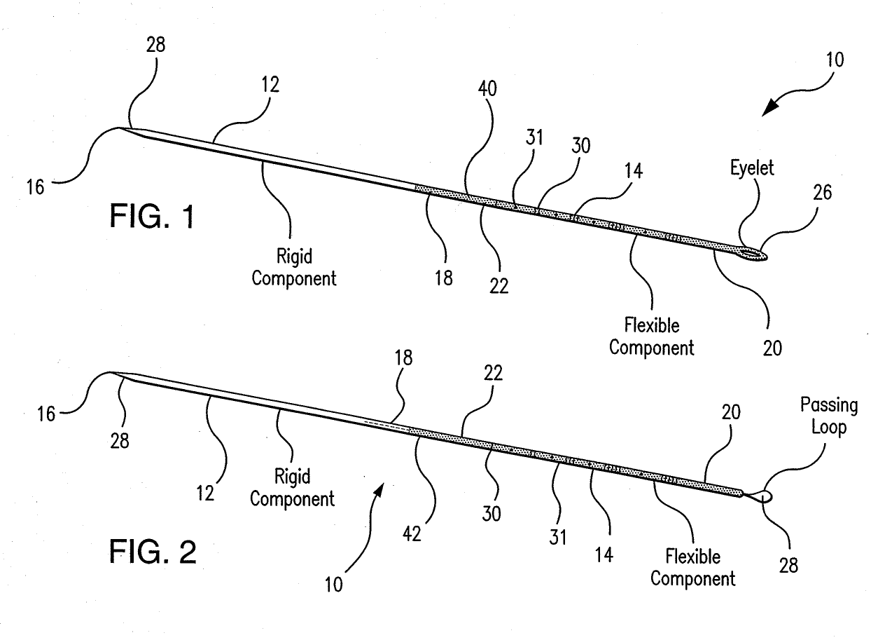

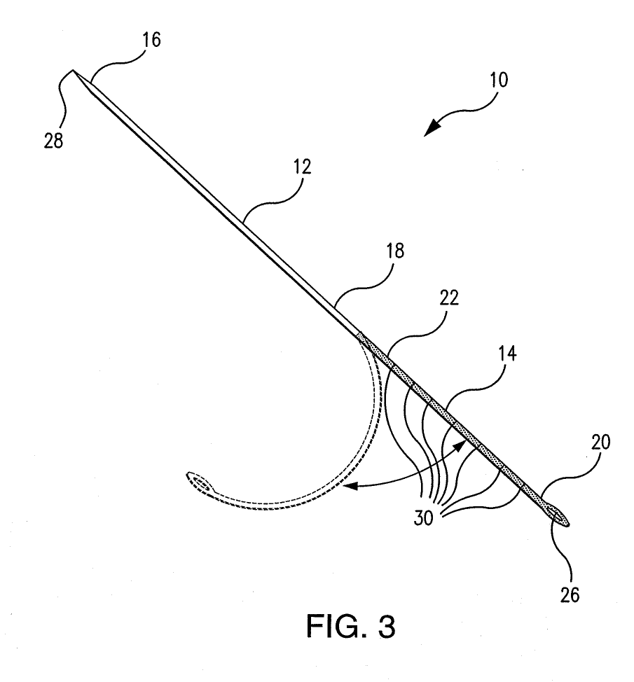

In accordance with the present invention, and with reference to FIGS. 1 to 9, a hybrid solid-flexible wire pin 10 and associated method for use in femoral tunnel reaming through an anteromedial portal during anterior cruciate ligament repair are disclosed. The hybrid solid-flexible wire pin 10 includes a leading portion 12 and trailing portion 14. The leading portion 12 includes a pointed, free first end 16 and a second end 18. Similarly, the trailing portion 14 includes a free first end 20 and a second end 22. The second ends 18, 22 of the respective trailing portion 12 and leading portion 14 are fixedly coupled to create a unitary hybrid solid-flexible wire pin 10. It is contemplated the hybrid solid-flexible wire pin 10 may be manufactured by either joining a solid leading portion and flexible trailing portion by melding them together 40 (as shown with reference to embodiment of FIG. 1) or by adjusting the properties of an integrally formed hybrid solid-flexible wire pin such that the leading and trailing portions exhibit desirable characteristics. In addition, the two ends may be joined together in a male/female arrangement 42 through a press-fit (see embodiment of FIG. 2) or may be joined together in any suitable manner.

The leading portion 12 is composed of a solid, substantially rigid member including a sharp tip 24 at the first end 16 thereof.

The trailing portion 14 is composed of a flexible component having an eyelet 26 (see embodiment shown in FIG. 1) or a passing loop 28 (see embodiment shown in FIG. 2) at the second end 22 thereof. The trailing portion 14 is further provided with distance markings 30, 31 which may be used in performing the procedure discussed below. The distance markings assist in preventing drilling over the solid leading portion and permit drilling only over the flexible trailing portion by allowing the surgeon to readily determine how much the flexible trailing portion remains in the bone. Depending on the procedure, surgeons choose various tunnel lengths and so if the surgeon wanted a length of a given distance, he would want to know that a greater portion of the flexible trailing portion is within the bone than the depth of the tunnel. It is contemplated markings will be provided to signify every 0.5 cm. In FIGS. 1 and 2, notches 30 are used to represent each centimeter and then a dot 31 is placed between them representing the half way mark between each marking.

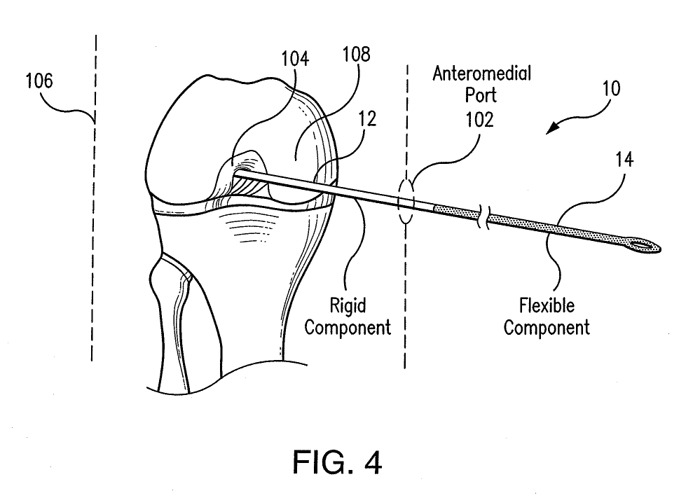

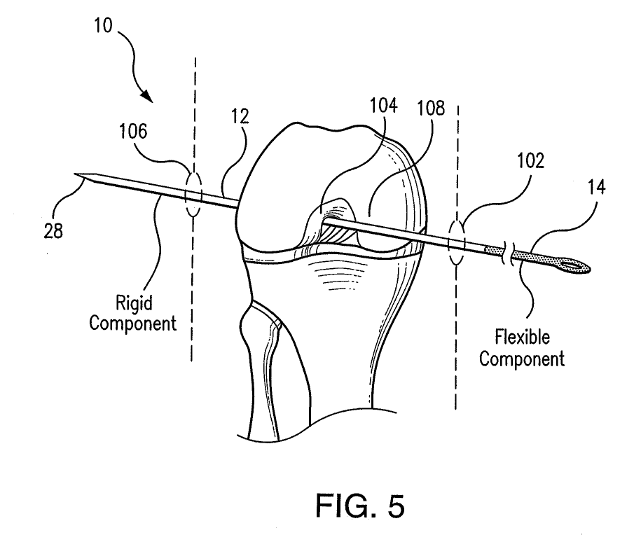

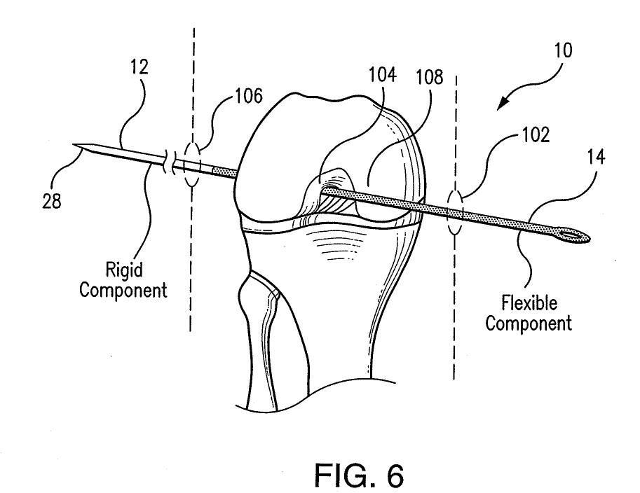

Briefly, and with reference to FIGS. 4 to 9, femoral tunnel reaming through an anteromedial portal 102 employing the present hybrid solid-flexible wire pin 10 during anterior cruciate ligament repair is disclosed. As shown in FIG. 4, the solid sharp leading portion 12 of the hybrid solid-flexible wire pin 10 is advanced through the anteromedial portal 102 to develop the femoral pilot hole utilizing a wire pin driver (not shown), for example, the CORE Universal Driver manufactured by Stryker Corporation, and the standard antegrade technique, that is, “pushing” the hybrid solid-flexible wire pin 10 into the femoral tunnel site 104 from the anteromedial portal 102, until the sharp tip 28 of the hybrid solid-flexible wire pin 10 exits the lateral femoral skin 106. The hybrid solid-flexible wire pin 10 is then advanced further (in a retrograde manner) by using the wire pin driver to “pull” the hybrid solid-flexible wire pin 10 until the trailing portion 14 exits the lateral skin 106 or is inserted to a depth greater than the desired femoral tunnel length as determined by markings on the trailing portion 14 of the hybrid solid-flexible wire pin 10 (see FIGS. 5 and 6).

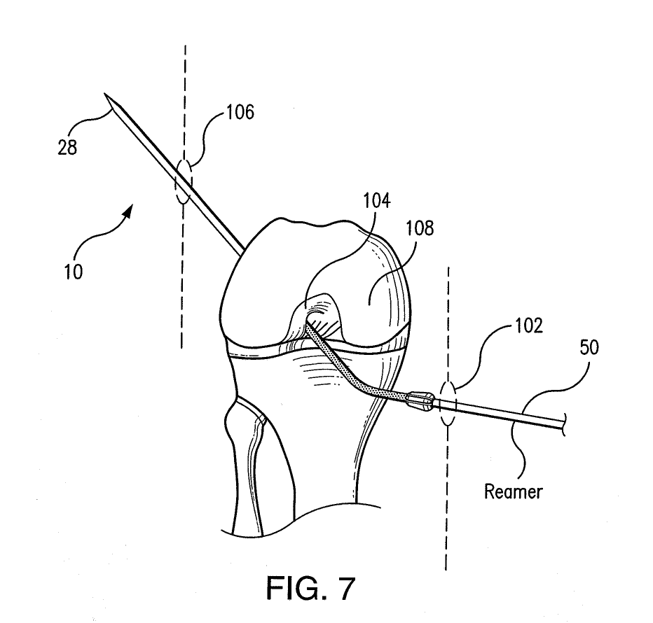

With the hybrid solid-flexible wire pin 10 in this position, and with reference to FIG. 7, a reamer 50 is passed over the hybrid solid-flexible wire pin 10 and through the anteromedial portal 102. The hybrid solid-flexible wire pin 10 is then temporarily deformed to allow the reamer 50 to clear the medial femoral condoyle 108 and the knee is hyperflexed to enable proper reaming.

Following the development of the femoral tunnel 110, the reamer 50 is removed from the femoral tunnel 110 and the knee is extended. Once again, by temporarily deforming the hybrid solid-flexible wire pin 10 the reamer 50 is directed away from the femoral condoyle and out of the anteromedial portal 102.

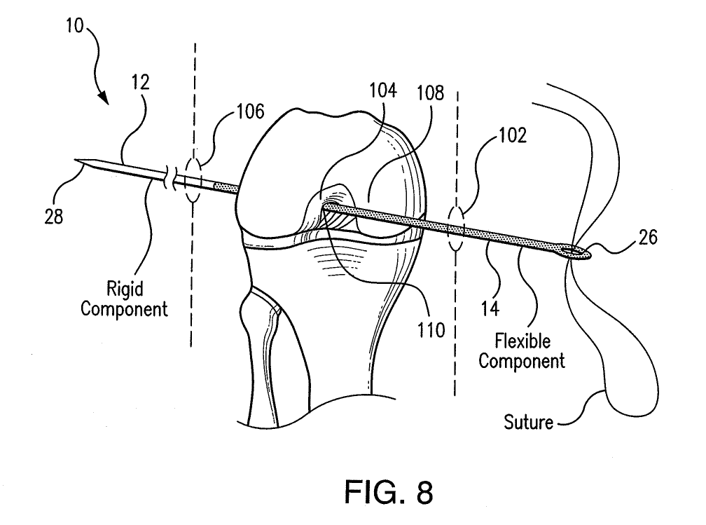

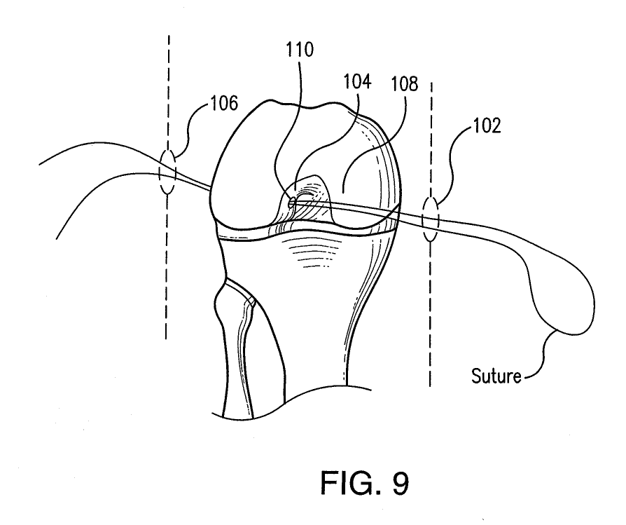

At the surgeon's choice, the eyelet 26 or passing loop 28 on the hybrid solid-flexible wire pin 10 could then be used to pass a passing suture loop 40 (see FIGS. 8 and 9) into the femoral tunnel 110 for later graft passage while removing the hybrid solid-flexible wire pin 10 out of the lateral thigh or this step could be performed later, using the same hybrid solid-flexible wire pin and technique.

The present hybrid solid-flexible wire pin can be utilized in any repair where there is little clearance for the pin in a bone tunnel, such as, for example, in arthroscopies requiring labral repair.

While the preferred embodiments have been shown and described, it will be understood that there is no intent to limit the invention by such disclosure, but rather, is intended to cover all modifications and alternate constructions falling within the spirit and scope of the invention.

1. A unitary hybrid solid-flexible wire pin, comprising:
a leading portion composed of a solid, substantially rigid member and a trailing portion composed of a flexible component,
the leading portion having a free first end and a second end,
the trailing portion having a free first end and a second end,
wherein the second end of the trailing portion and the second end of the leading portion are fixedly coupled to create the unitary hybrid solid-flexible wire pin.

2. The unitary hybrid solid-flexible wire pin as claimed in claim 1, wherein the second end of the trailing portion has an eyelet or a passing loop therein.

3. The unitary hybrid solid-flexible wire pin as claimed in claim 1, wherein the trailing portion is provided with distance markings thereon.

4. The unitary hybrid solid-flexible wire pin as claimed in claim 3, wherein the distance markings are provided at 0.5 cm intervals.

5. The unitary hybrid solid-flexible wire pin as claimed in claim 1, wherein the distance markings are placed at 1 cm intervals with a notch and 0.5 intervals with a dot.

6. The unitary hybrid solid-flexible wire pin as claimed in claim 1, wherein the trailing portion is composed of a nickel titanium shape memory alloy.

7. The unitary hybrid solid-flexible wire pin as claimed in claim 1, wherein the leading portion and the trailing portion are joined by melding or by a press-fit male/female arrangement.

8. The unitary hybrid solid-flexible wire pin as claimed in claim 1, wherein the free first end of the leading end includes a sharp tip.

9. A method for femoral tunnel reaming through an anteromedial portal during anterior cruciate ligament repair using an unitary hybrid solid-flexible wire pin, the unitary hybrid solid-flexible wire pin comprising a leading portion composed of a solid, substantially rigid member and a trailing portion composed of a flexible component,
the leading portion having a free first end and a second end, the trailing portion having a free first end and a second end, wherein the second end of the trailing portion and the second end of the leading portion are fixedly coupled to create the unitary hybrid solid-flexible wire pin, the method comprising:
advancing the free first end of the leading edge portion of the unitary hybrid solid-flexible wire pin through the anteromedial portal to develop a femoral pilot hole;
further advancing the unitary hybrid solid-flexible wire pin in a retrograde manner until the trailing portion is inserted to a depth greater than a desired femoral tunnel length;
passing a reamer over the unitary hybrid solid-flexible wire pin and through the anteromedial portal;
deforming the unitary hybrid solid-flexible wire pin to allow the reamer to clear a medial femoral condoyle while hyperflexing a knee;
removing the reamer from the femoral tunnel and extending the knee;
temporarily deforming the unitary hybrid solid-flexible wire pin to enable the reamer to be directed away from the femoral condoyle and out of the anteromedial portal.

10. The method, according claim 9, further comprising the step of passing a suture loop into an aperture in the trailing portion of the unitary solid-flexible wire pin and thereby into the femoral tunnel for graft passage while removing the unitary hybrid solid-flexible wire pin.

11. The method, according to claim 9, wherein the step of advancing the unitary hybrid solid-flexible wire pin in a retrograde manner until the trailing portion is inserted to a depth greater than a desired femoral tunnel length is achieved by use of distance markings located on the trailing portion of the unitary hybrid solid-flexible wire pin.
